(19) United States
(12) Patent Application Publication
(10) Pub. No.: US 2015/0104404 A1
(43) Pub. Date: Apr. 16, 2015

(54) POLYMER SYNERGY FOR IMPROVED HAIR STYLING

(71) Applicant: The Dial Corporation, Scottsdale, AZ (US)

(72) Inventor: Jennifer Harris, Phoenix, AZ (US)

(73) Assignee: The Dial Corporation, Scottsdale, AZ (US)

(21) Appl. No.: 14/054,805

(22) Filed: Oct. 15, 2013

(51) Int. Cl. A61K 8/81; A61Q 5/06

(52) U.S. Cl. CPC A61K 8/8158; A61K 8/8135; A61Q 5/06
USPC 424/70.15; 424/70.16

(57) ABSTRACT

A hair styling composition comprises: 5 wt. % to 50 wt. % of a first nonionic polymer; 2 wt. % to 20 wt. % of a second nonionic polymer; and 2 wt. % to 15 wt. % of a cationic polymer. A method of making a hair styling composition comprises: combining 5 wt. % to 50 wt. % of a first nonionic polymer, 2 wt. % to 20 wt. % of a second nonionic polymer, and 2 wt. % to 15 wt. % of a cationic polymer to form a hair styling composition.

### TECHNICAL FIELD

The present disclosure relates generally to synergy between polymers that provide improved hold in styling products over other combinations of styling polymers.

### BACKGROUND

Most hair styling products comprising a polymer or various combinations of polymers generally need to increase the amount of polymer used in the hair styling product or to increase the amount and/or number of polymers used in the hair styling product in order to increase various properties of the hair styling product including the hold attribute. It can be desirable in personal grooming to maintain one's hair in a particular setting, and a common method of accomplishing this is to apply a hair styling composition to the hair and allowing it to dry and set either on its own or with heat from a hair dryer or hot curlers. Hair setting compositions can assist in styling the hair and can also provide a temporary hold to the hair while imparting shine and appearance benefits.

Hair styling products can be marketed in various forms, including hair spray, styling gel, setting foam, or hair lacquer. The objective in formulating hair styling gels has always been to optimize a number of interrelated physical properties of the product seen both before and after drying. These parameters include a desirable viscosity for the product such that it dispenses nicely (e.g., when squeezed from a plastic tube) and feels acceptable in the hands and hair when applied to the hair. Also desirable is to balance the final level of hold when dry with the ability to wash out again with shampoo, and to minimize flaking. These parameters are evaluated by various visual and tactile sensory methods including viewing, touching, combing/brushing, looking at shine, cleanliness, stiffness, tackiness, bounce, ability to re-style, combing ease, residue/flaking, static, smoothness and the like.

Hair styling compositions generally include hair setting polymers (styling or fixative), natural gums and resins, viscosifier polymers, polymer modifiers, solvents, co-solvents, colors, fragrances, preservatives and the like. Styling and fixative polymers typically include nonionic and anionic fixative polymers, also known as film former polymers. Among the most commonly used styling and fixative polymers and copolymers are mixtures of vinyl acetate and vinylpyrrolidone along with anionic polymers or combinations of anionic polymers with nonionic or cationic polymers. Some commercially available polymers that have been used include polyvinylpyrrolidone (PVP), sold under the trade name of Luviskol-PVP from BASF, polyvinylpyrrolidone/vinylacetate copolymer (PVP/VA), sold under the trade name Luviskol-VA® from BASF, vinyl pyrrolidone/dimethylaminoethyl methacrylate copolymer, sold under the trade name of Styleze® from ISP, vinyl caprolactam/vinyl pyrrolidone/dimethylaminoethyl methacrylate copolymer with 0.77% ethanol, sold under the trade name of Advantage®-S Solution from ISP, and vinyl caprolactam/vinyl pyrrolidone/dimethylaminoethyl methacrylate copolymer with 63% ethanol, sold under the trade name of Advantage® LC-A from ISP.

When hair setting resins and fixative polymers alone cannot provide the desired viscosity for the hair setting product, formulators may add rheology modifiers or gellants. These ingredients may include polymers, gums or resins, or other rheology modifiers such as emulsifiers, waxes, and the like, to achieve the target rheology for the product.

When a hair setting composition is dried on the hair, it is important to optimize strength and elasticity of the film on the hair, and to ensure the dried film on each hair shaft does not flake or dust when the hair is subject to stress throughout the day, such as combing. Additionally, the set hair should be tack-free, clear, glossy, humidity-resistant, yet still be able to be easily washed out of the hair with shampoo products. To achieve these desirable attributes, it is common to blend both synthetic copolymers of the type mentioned above with carbohydrate or starch-based materials such as hydroxyethylcellulose and/or modified corn starch, the latter sold under the trade name Amaze® from National Starch. Such combinations of starches and their derivatives with synthetic vinyl copolymers are thoroughly mentioned in U.S. Pat. No. 6,413,505 issued to Vitale et al. and incorporated herein by reference. Other ingredients that help optimize the physical properties of hair gel include emulsifiers, petrolatum, and various fatty alcohols.

Most hard-hold hair styling products rely on high concentrations of polymers and copolymers to provide a desirable stiff feel and high level of hold. Many polymers are typically supplied as ethanolic solutions so that the end formulator can use a fairly concentrated raw material that still can be poured into the mixer. New regulatory initiatives to improve air quality have centered on reduction of volatile organic carbons (VOC's) in many household and industrial products, and these regulations have affected the formulation of personal hair care products. For example, regulations in the state of California will eventually limit VOC level in hair care holding gels to 2% maximum. If a formulator simply substitutes aqueous polymer solutions for the ethanolic solutions, the resulting compositions impart less stiffness in the hair and the hold is dramatically reduced. Formulators have tried to compensate for this lack of hold by simply increasing the amount of the polymers. However, simply increasing the amounts of various polymers in an aqueous formula will not improve the formula's hard-hold characteristics. Such a strategy merely increases the cost of the formulation and increases brittleness and flaking of the dried composition in the hair. Consequently it is desirable to formulate low- to no-VOC hair gels that have an absence of ethanolic solutions of raw materials, but still have consumer acceptable characteristics. More particularly, what is needed is a way to formulate low- to no-VOC hair styling compositions that still provide consumer acceptable feel, hold, and shine without undue flaking.

Thus, what is needed are hair styling products comprising a lower amount of polymer(s) and/or fewer polymers with increased hair styling properties such as hold attribute.

Furthermore, other desirable features and characteristics of the polymers and styling products disclosed herein will become apparent from the subsequent detailed description and the appended claims, taken in conjunction with the accompanying drawings and this background.

### BRIEF SUMMARY

Disclosed herein, in various embodiments, are hair styling compositions, methods for making the hair styling compositions, and articles comprising the hair styling compositions.

In an embodiment, a hair styling composition comprises: 5 wt. % to 50 wt. % of a first nonionic polymer; 2 wt. % to 20 wt. % of a second nonionic polymer; and 2 wt. % to 15 wt. % of a cationic polymer.

In an embodiment, a method of making a hair styling composition comprises: combining 5 wt. % to 50 wt. % of a first nonionic polymer, 2 wt. % to 20 wt. % of a second nonionic polymer, and 2 wt. % to 15 wt. % of a cationic polymer to form a hair styling composition.

### DETAILED DESCRIPTION

The following detailed description is merely exemplary in nature and is not intended to limit the disclosed herein or the application and uses of the compositions and/or products disclosed herein. Furthermore, there is no intention to be bound by any theory presented in the preceding background or the following detailed description.

Rather, the following description provides a convenient roadmap for implementing exemplary embodiments of the compositions and/or products disclosed herein. Various changes to the described embodiments may be made in the function and relative amounts of the ingredients described without departing from the scope of the invention as set forth herein. Additionally, though demonstrated herein in terms of water-based, non-aerosol hair setting gel compositions with relatively high viscosity, other product forms the present invention may take include hair sprays, pourable liquids, sprays, spritzes, foams, crèmes, pastes, non-runny gels, mousses, pomades, lacquers, non-aerosol pump-spray liquids and the like. Furthermore, the compositions of the present invention may include adjuvants such as deodorants, fragrances, aromatherapy essences, herbs, infusions, antimicrobials, pediculicides, stimulants, essential oils, hair coloring, dying or tinting agents, anti-gray agents, sun-blockers, vitamins, antioxidants, surfactants and other wetting agents, Rogaine®, mica, silica, metal flakes or other glitter-effect materials, conditioning agents, anti-static agents, opacifiers, detackifying agents, penetrants, preservatives, and emollients and the like, and these compositions having additional consumer benefits (perceivable or otherwise) likewise fall within the ambit of the compositions and/or products disclosed herein.

Disclosed herein are hair styling products comprising a combination of polymers resulting in an unexpected synergy between the polymers thereby providing an increase in hair styling properties of the hair styling products such as an increase in the hold attribute over other combinations of styling polymers. For example, the hair styling products disclosed herein can comprise a combination of greater than or equal to three polymers including polyvinylpyrrolidone (PVP), a vinylpyrrolidone/dimethylamino propylacrylamide (DMAPA) acrylates copolymer, and/or a vinyl caprolactam/vinylpyrrolidone/dimethylaminoethyl methacrylate copolymer. The combination of these polymers can result in an unexpected synergistic effect with increased hair styling attributes, such as an increase in the hold attribute as compared to hair styling products comprising other polymers. The combination of these polymers can yield a synergistic matrix thereby forming a high hold hair styling product.

That being said, the hair styling products disclosed herein provide compositions useful for styling and setting hair that comprise polymers, copolymers, starches and, polyols. Most remarkable, these compositions are low- to no-VOC yet still impart hard-hold to hair when dried on the hair, and this is possible by incorporating various polyols that improve the hardness and durability of the resulting film on the hair. The compositions disclosed herein can comprise; (a) a synthetic polymer or copolymer; (b) a starch or other cellulosic material, natural or modified; (c) water; and, (d) a polyol, wherein the final composition contains less than 2.0 weight percent (wt. %) volatile organic compounds (VOC).

Additionally, the compositions disclosed herein can include viscosity modifying polymers including cellulosic materials (for example for thickening), an oil phase comprising petrolatum or the like, fatty alcohols, emulsifiers, and various oil-soluble film formers, optional solvents and co-solvents selected from the group considered “VOC-exempt” (acetone, d-limonene and the like), and any combination of the adjuvants mentioned previously to either impart marketable benefits or to adapt the basic formulations to a particular physical product form and delivery method/preferred packaging, (liquid, gel, pump, aerosol, etc.).

Multiplying each of the solvent levels present in the individual raw materials by the amount of that raw material used in the composition, and then adding these fractional amounts, give the “VOC content” or “percent volatile organic compounds” in a composition. Ethanol, for example, may be 100% VOC contributing raw material (when absolute alcohol), whereas some raw materials, such as water, various powdered polymers and starches, or water based solutions of polymers, may contribute no VOC at all to the composition. Frequently however, synthetic polymers are obtained in alcoholic solution (e.g., 20 to 80 wt. % solvent) and using these ingredients adds to the VOC content of the final composition.

Polyols as used herein can include but are not limited to straight-chain molecules having carbon chain length of about 3 to about 8, specifically, about 5 to about 6, with the number of hydroxyl groups of about 3 to about 8, specifically, about 5 to about 6. “Polyols” as described herein are understood to be substances with 3 or more hydroxyl groups (i.e., including triols but excluding diols). For example, straight chain polyols can include erythritol, glycerol, mannitol, sorbitol, xylitol, and combinations comprising at least one of the foregoing. The polyols can be naturally or synthetically derived, and can also include cyclic, or combinations of straight chain and cyclic structures. For example, isomalt, lacitol, maltitol, and various HSH's (hydrogenated starch hydrosylates) can be used. For example, mannitol or sorbitol (both C₆H₁₄O₆) or combinations of the two can be used. Polyols that contain, in addition to the desired minimum (3) hydroxyl groups, other functional groups in the molecule such as aldehydes, ketones, carboxylate, thiols, etc. can also be used in the hair styling products disclosed herein. For example, methyl 2,5,6-trihydroxyhexanoate, or other functionalized polyols can find use herein. Additionally, natural sugar monosaccharides that are in equilibrium mixture of open straight chain form and cyclic aldol or ketol form can also be used. Many small organic molecules, or mixtures thereof, can also be used herein to provide hard-hold provided they minimally have three or more hydroxyl groups. The polyol or polyols can be incorporated in the compositions and/or hair styling products disclosed herein in amounts of about 5 wt. % to about 15 wt. % by weight in the composition. It can also be desirable to maintain a ratio of polyol to total synthetic and natural polymers of about 1:3 to about 1:10.

The polymer or copolymer used in the hair styling products disclosed herein can be nonionic, anionic, zwitterionic or amphoteric or cationic synthetic homopolymers, and copolymers. For example, nonionic polymers can include polyvinylpyrrolidone (PVP), copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, copolymers of vinylpyrrolidone (VP) and dimethylamino propylacrylamide (DMAPA), terpolymers of vinylcaprolactam, vinylpyrrolidone and dimethylaminoethyl methacrylate, polysiloxanes, and combinations comprising at least one of the foregoing.

Anionic polymers include vinyl acetate/crotonic acid, vinyl acetate/acrylate and/or vinyl acetate/vinyl neodecanoate/crotonic acid copolymers, sodium acrylate/vinyl alcohol copolymers, sodium polystyrenesulfonate, ethyl acrylate/-N-tert-butylacrylamide/acrylic acid copolymers, vinylpyrrolidone/vinylacetate/itaconic acid copolymers, acrylic acid/acrylamide copolymers and/or sodium salts thereof, homo- and/or copolymers of methacrylic acid and/or salts thereof, and acrylate/hydroxyacrylate, octylacrylamide/acrylate or methacrylate and/or butyl acrylate/N-vinylpyrrolidone copolymers or polystyrenesulfonates.

Amphoteric polymers include copolymers of N-octylacrylamide, methacrylic acid and tert-butylaminoethyl methacrylate of the “amphomer” type, copolymers of methacryloylethylbetaine an alkylmethacrylates of the “yukaformer” type, copolymers of monomers containing carboxyl groups or sulfone groups, for example methacrylic acid and itaconic acid, with basic group-containing monomers such as mono- or dialkylaminoalkyl methacrylates and/or mono- and dialkylaminoalkyl methacrylamides, copolymers of N-octylacrylamide, methyl methacrylate, hydroxypriopyl methacylate, N-tert-butylaminoethyl methacrylate and acrylic acid.

Cationic polymers include vinylpyrrolidone/vinylimidazolium methochloride copolymers, quaternized vinylpyrrolidone/diakylaminoalkyl methacrylate copolymers, cationic cellulose derivatives, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers.

That being said, the hair styling products disclosed herein can comprise compositions comprising vinyl pyrrolidone/dimethylaminoethyl methacrylates copolymer, vinyl acetate/crotonates/vinyl neodecanoate copolymer, octyl acrylamide/acrylates/butylaminoethyl methacrylate copolymer, vinyl acetate/crotonates, polyvinylpyrrolidone (PVP), polyvinylpyrrolidone/vinyl acetate copolymer, PVP acrylates copolymer, VP/DMAPA acrylates copolymer, vinyl caprolactam, vinyl acetate/crotonic acid/vinyl proprionate, acrylates/acrylamide, acrylates/octylacrylamide, acrylates copolymer, acrylates/hydroxyacrylates copolymer, and alkyl esters of polyvinylmethylether/maleic anhydride, diglycol/cyclohexanedimethanol/isophthalates/sulfoisophthalates copolymer, vinyl acetate/butyl maleate and isobornyl acrylate copolymer, vinylcaprolactam/PVP/dimethylamino ethyl methacrylate, vinyl acetate/alkylmaleate half ester/N-substituted acrylamide terpolymers, vinyl caprolactam/vinylpyrrolidone/dimethylaminoethyl methacrylates copolymer, vinyl caprolactam/vinylpyrrolidone/methacryloamidopropyl trimethylammonium chloride terpolymer, methacrylates/acrylates copolymer/amine salt, polyvinylcaprolactam, polyurethanes, polyquaternium-4, polyquaternium-10, polyquaternium-11, polyquaternium-46, hydroxypropyl guar, hydroxypropyl guar hydroxypropyl trimmonium chloride, polyvinyl formamide, polyquaternium-7, and hydroxypropyl trimmonium chloride guar.

The polymers for use in the hair styling products and compositions disclosed herein can include in various ratios and amounts of polyvinylpyrrolidone, polyvinylpyrrolidone/vinyl acetate copolymer, vinyl pyrrolidone/dimethylaminoethyl methacrylates copolymer, and vinyl caprolactam/vinyl pyrrolidone/dimethylaminoethyl methacrylates copolymer. The preferred total amount of synthetic fixative polymers is about 30 wt. % to about 70 wt. %, with either one of these or combinations thereof. In order to keep the VOC level in the composition at or below 2%, it is important to use polymers that are available in aqueous or nearly aqueous solution, or in powdered form, rather than using polymers supplied in solvent solutions that will contribute to the VOC content of the final composition. The polyvinylpyrrolidone polymer can be present in an amount of 5 weight percent (wt. %) to 50 wt. %, specifically, 10 wt. % to 40 wt. %, more specifically, 15 wt. % to 30 wt. %, and even more specifically, 20 wt. % to 25 wt. %. The vinylpyrrolidone/DMAPA acrylates copolymer can be present in an amount of 2 wt. % to 20 wt. %, specifically, 5 wt. % to 15 wt. %, and more specifically, 8 wt. % to 13 wt. %. The vinyl caprolactam/vinyl pyrrolidone/dimethylaminoethyl methacrylate copolymer can be present in an amount of 2 wt. % to 15 wt. %, specifically, 5 wt. to 10 wt. %, and more specifically 9 to 12 wt. %. Use of these polymers in the amounts described can provide a surprising synergy resulting a hair styling composition having increased hold attributes as compared to a hair styling composition comprising different or an increased number of polymers.

For example, the compositions can include polyvinylpyrrolidone 20% aqueous solution, or polyvinylpyrrolidone powder, sold under the trade names of Luviskol K-90® and Luviskol-PVP® respectively commercially available from BASF (both 0% VOC contribution), vinyl pyrrolidone/dimethylaminoethyl propylacrylamide acrylates copolymer (VP/DMAPA acrylates copolymer-copolymer), sold under the trade name of Styleze CC-10® commercially available from ISP (0% VOC contribution), and vinyl caprolactam/vinyl pyrrolidone/dimethylaminoethyl methacrylate copolymer with 0.77% ethanol, sold under the trade name of Advantage®-S Solution from ISP (0.77% VOC contribution). The PVP powder or solution can be present in an amount of 15 wt. % to 30 wt. %, the VP/DMAPA acrylates copolymer can be present in an amount of 8 wt. % to 13 wt. %, and the vinyl caprolactam/VP/dimethylaminoethyl methacrylate copolymer can be present in an amount of 9 wt. % to 12 wt. %, based on the weight of the composition.

The compositions disclosed herein can also include starch, modified starches and/or other cellulosic material or combinations of these, for modifying the quality of the dried film on the hair and for modifying the viscosity of the actual composition. For example, of use in the compositions disclosed herein include celluloses, cellulose derivatives, cellulose gums, ethoxylated celluloses, starch or gums, guar gum, guar hydroxypropyl trimonium chloride, xanthan gum, hydroxypropyl guar, karaya gum, as well as combinations comprising at least one of the foregoing. Also of use in the compositions disclosed herein are pregelatinized crosslinked starch derivatives, including hydroxypropyl distarch phosphate, as described in U.S. Patent Application Publication US2005/0191264 and incorporated herein by reference. Cellulosic materials for use herein can include nonionic or cationic saccharides such as cellulose ethers including methyl cellulose, carboxymethyl cellulose, hydroxy propyl methyl cellulose, hydroxyethyl cellulose, hydroxypropyl cellulose, and ethyl hydroxyethyl cellulose, dextrans obtained from Sigma, Kitamer PC, a chitosan carboxylate and Kytamer L, a chitosan lactate obtained from Amerchol, Gafquat HS-100, Polyquatemium-28 from International Specialties, polyquaternium4, polyquaternium-10, sodium alginate, agarose, amylopectins, amyloses, arabinans, arabinogalactans, arabinoxylans, carrageenans, gum arabic, cellulose derivatives such as methylcellulose, hydroxypropylmethylcellulose, hydroxyethyl cellulose, carboxymethylcellulose, carboxymethylguar gum, carboxymethyl(hydroxypropyl)guar gum, hydroxyethylguar gum, hydroxypropylguar gum, cationic guar gum, chondroitins, chitins, chitosans, cocodimonium hydroxypropyl oxyethyl cellulose, colominic acid [poly(N-acetyl-neuraminic acid], corn starch, curdlan, dermatin sulfate, furcellarans, dextrans, cross-linked dextrans known as dextranomer (Debrisan), dextrin, emulsan, flaxseed saccharide (acidic), galactoglucomannans, galactomannans, glucomannans, glycogens, guar gum, or hydroxyethylstarch, hydroxypropylstarch, hydroxypropylated guar gums, gellan gum, glucomannans, gellan, gum ghatti, gum karaya, gum tragacanth (tragacanthin), heparin, hyaluronic acid, inulin, keratan sulfate, konjac mannan, laminarans, laurdimonium hydroxypropyl oxyethyl cellulose, liposan, locust bean gum, mannans, nigeran, nonoxylnyl hydroxyethyl cellulose, okra gum, oxidized starch, pectic acids, pectins, polydextrose, potato starch, protopectins, psyllium seed gum, pullulan, sodium hyaluronate, steardimonium hydroxyethyl cellulose, raffinose, rhamsan, tapioca starch, welan, levan, scleroglucan, stachyose, succinoglycan, wheat starch, xanthan gum, xylans, xyloglucans, and mixtures thereof. Microbial saccharides can be found in the fourth edition of Kirk-Othmer Encyclopedia of Chemical Technology, Fourth Edition. Vol. 16, John Wiley and Sons, NY pp. 578-611, 1994. Complex carbohydrates can be found in the fourth edition of Kirk-Othmer Encyclopedia of Chemical Technology, Fourth Edition. Vol. 4, John Wiley and Sons, NY pp. 930-948, 1994. For example, hydroxyethylcellulose and a modified corn starch from National Starch found under the trade name Amaze® can be used. The compositions disclosed herein can include about 0.01 wt. % to about 10 wt. % total cellulosic and starch materials, by weight in the composition. For example, hydroxyethylcellulose and Amaze® modified corn starch from National Starch can be used, together in the composition at a total of about 0.1 wt. % to about 5 wt. % by weight.

The compositions can also include a thickening polymer, such as a high molecular weight polyacrylate. Thickeners can be utilized alone or in combination so long as the chosen thickeners are compatible with the hair styling composition. Thickeners may include, but are not limited to, acrylic acid homopolymers under the Carbopol® trade name from BF Goodrich, acrylates/C10-30 alkyl acrylate crosspolymer (Carbopol® 1342, 1382, Pemulins® TR-1 and TR-2 from BF Goodrich), Acrylates/Steareth-20 Itaconate Copolymer, Acrylates/Ceteth-20 Itaconate Copolymer from National Starch, Bentonite, PVM/MA Decadiene Crosspolymer from International Specialties Products, Acrylates/steareth-20 methacrylate copolymer, Acrysol® ICS-1 from Rohm and Haas Co., acrylamide/sodium acrylate copolymer, Hostacerin® PN 73, Hoecsht A G., acrylate copolymer (Antil® 208) supplied by Degussa-Goldschmidt, acrylic acid/acrylonitrogens copolymer (Hypan® SA-100H, SR-150H) supplied by Lipo, Acrylic/acrylate copolymer (Carboset® 514, 515, 525, XL-19, XL-19X2, XI-28, XL40, 526) supplied by BF Goodrich, Ammonium acrylateslacrylonitrogens copolymer (Hypan® SS-201) from Lipo, Quaternium-18 Bentonite, Sodium salt of crosslinked poly(acrylic acid) under the tradenames PNC® 430, PNC® 410, PNC® 400 from 3V, Stearalkonium Bentonite, Claytone, supplied by Southern Clay, Quatemium-18 Hectorite (Bentone 38), Stearalkonium Hectorite (Bentone 27) supplied by Rheox, acrylamide/sodium acrylate copolymer (Hostacerin PN 73) supplied by Hoechst, Poly(acrylic acid) known as Carbopol® 400 series (BF Goodrich) or Aquatreat® (Alco 3V), polyquaternium-18 (Mirapol® AZ-1) from Rhone Poulenc, polyquaternium-27, polyquaternium-31, polyquaternium-37, trihydroxystearin (Thixcin from Rheox; Flowtone from Southern Clay), Dimethylaminoethyl methacrylamide and acrylamide copolymer (Salcare® SC63 from Ciba Specialties), Acrylic polymer cationic thickening agents (Synthalen® CR and its related compounds) from 3V Sigma. Other thickeners and polymers can be found in the “The Encyclopedia of Polymers and Thickeners for Cosmetics,” Cosmetics and Toiletries, Lochhead, R., pp. 95-138, Vol. 108, (May 1993). Thickeners, when present, can be incorporated in amounts of about 0.01 wt. % to bout 5 wt. % by weight active polymer.

The compositions can also include petrolatum or other waxes and oils. Petrolatum is a mixture of hydrocarbons that finds use in various personal care products. The petrolatum used in the compositions disclosed herein can include white petrolatum USP, petrolatum USP, mineral jelly, and ointment base. The melting point ranges of the preferred petrolatum for use in the present invention can be about 80° F. to about 135° F. For example, UltraPure® Liquid Petrolatum USP from Ultra Chemicals that has a melting point range of about 105°-115° F., or various grades (ranging in color) of Penreco® Petrolatum USP having melting point ranges around 122°-135° F. can be used. The compositions can also contain an emulsifying wax and/or oil. Such materials include the non-limiting examples of bees' wax, candelilla wax, carnauba wax, emulsifying wax (for example Polawax® from Croda) and Jojoba, safflower, canola (tribehenin), tallow, lard, palm, castor, sunflower seed, or soya bean oil oils, or hydrogenated derivatives thereof. Most preferred is to incorporate Polawax®, jojoba oil, safflower oil, tribehenin, and/or hydrogenated castor oil, singularly, or in any combination. When desired in the present compositions, any combination of these materials can be used in an amount of about 0.1 wt. % to about 5 wt. % by weight of the total composition. For example, petrolatum, wax, and/or oil in combination or individually, can be used in an amount of about 1 wt. % to about 3 wt. % by weight in the composition.

Fatty alcohols that may find use in the compositions disclosed herein can include naturally derived and synthetic materials. These are high molecular weight straight or branched chain primary alcohols. For example, the fatty alcohol can be lauryl (C12), myristyl (C14), cetyl or palmityl (C16), stearyl (C18), oleyl (C18-unsaturated) and linoleyl (C18-polyunsaturated) alcohols, or combinations thereof. Ceteryl alcohol can also be used and is a mixture of cetyl and stearyl alcohols. The fatty alcohol, when present, can be incorporated in an amount of about 0.01 wt. % to about 5 wt. % by weight in the composition.

Also of use as an optional ingredient in the compositions disclosed herein are emulsifiers. Emulsifiers for use in cosmetic applications are amply listed in McCutcheon's Emulsifiers and Detergents. Many emulsifiers are nonionic esters or ethers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl, or arylalkyl moiety, normally containing from about 8 to about 50 carbons. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil, sunflower seed oil, or soya bean oil. Such nonionic surfactants can also be derived from a polyhydroxy compound such as glycerol or sorbitol or other alditols. Examples of such emulsifiers include ceteareth-10 to -25, ceteth-10-25, steareth-10-25 (i.e. C₁₆ to C₁₈ alcohols ethoxylated with 10 to 25 ethylene oxide residues) and PEG-15-25 stearate or distearate. Other suitable examples include C₁₀-C₂₀ fatty acid mono, di- or tri-glycerides. Further examples include C₁₈-C₂₂ fatty alcohol ethers of polyethylene oxides (8- to 12-EO). Other examples of useful emulsifiers are fatty acid mono or possibly diesters of polyhydric alcohols such as glycerol, sorbitol, erythritol, or trimethylolpropane. The fatty acyl moiety is often from C₁₄ to C₂₂ and is saturated in many instances, including cetyl, stearyl, arachidyl, and behenyl. Examples include monoglycerides of palmitic or stearic acid, sorbitol mono or diesters of myristic, palmitic, or stearic acid, and trimethylolpropane monoesters of stearic acid. Another usable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers often terminate in C₁ to C₁₂ alkyl groups. Such emulsifiers and co-emulsifiers are widely available under many trade names and designations including Abil®, Arlacel®, Brij®, Cremophor®, Dehydrol®, Dehymuls®, Emerest®, Lameform®, Pluronic®, Prisorine®, Quest PGPR®, Span® Tween®, SF1228, DC3225C and Q2-5200.

Desirable emulsifiers can include any combination of fatty alcohols (such as mentioned previously), phosphate-based emulsifying waxes, sorbitan monooleates and stearates and other carbohydrate esters of fatty alcohols and their ethoxylated derivatives, and the polyalkylene glycols and polyethoxylated waxes. For example, a fatty alcohol blend for optional use in the present compositions is Crodafos® CES (white solid or flakes) from Croda, which is a blend of ceteryl alcohol, dicetyl phosphate and ceteth-10 phosphate. Apifil PEG-8 beeswax emulsifier, which is a combination of fatty acid esters and polyethylene glycol and is a nonionic self-emulsifying base can also be used. When desired in the composition, the emulsifier can be present in an amount of about 0.01 wt. % to about 10 wt. % by weight in the composition.

Also, the compositions disclosed herein can include “oil-soluble film former” polymers. Non-limiting examples of the oil-soluble film former include polymethylsilsesquioxanes; acrylic fluorinated emulsion film formers, such as Foraperle® film formers (e.g., Foraperle® 303 D available from Elf Atochem); GANEX® copolymers, such as butylated PVP, PVP/Hexadecene copolymers, PVP/Eicosene copolymers, and tricontanyl; Poly-(vinylpyrrolidone/diethylaminoethyl methacrylate) copolymers and PVP/Dimethylaminoethylmethacrylate copolymers such as Copolymer 845 available from I.S.P.; Resin ACO-5014 (Imidized IB/MA copolymer); other PVP based polymers and copolymers; silicone gums; cyclomethicone copolymers and dimethicone crosspolymers, such as Dow Corning® 2-9040 and those disclosed in U.S. Pat. No. 5,654,362, the disclosure of which is hereby incorporated by reference; trimethyl siloxysilicates such as SR 1000, 554230, and SS4267 available from GE Silicones; alkyl cycloalkylacrylate copolymers, such as those disclosed in WO 98/42298, the disclosure of which is hereby incorporated by reference; Mexomere® film formers and other allyl stearate/vinyl acetate copolymers; polyolprepolymers, such as PPG-12/SMDI copolymer, also called Poly-(oxy-1,2-ethanediyl), α-hydro-ω-hydroxy-polymer with 1,1′-methylene-bis-(4-isocyanatocyclohexane) available from Barnet; and Avalure® AC Polymers (Acrylates Copolymer) and Avalure® UR polymers (Polyurethane Dispersions), available from BFGoodrich. Preferred for use in the present invention is the already mentioned tricontanyl PVP copolymer sold under the trade name Ganex® WWP-660 from ISP. Any combination of the Ganex® V/WP grades can be used in the compositions disclosed herein and provide water and wear resistance and a moisture barrier to the set hair. The oil-soluble film former polymer can be present in the composition in an amount of about 0.1 wt. % to about 5 wt. % by weight.

Also optional to the compositions disclosed herein is the addition of a chelant. Chelants that can find use herein include but are not limited to phosphates (organic and inorganic), NTA, the various ethylenediaminetetraacetic acid (EDTA) derivatives, and lower molecular weight polyacrylates. For example, the present invention may include trisodium or tetrasodium EDTA, various salts of NTA, phosphate esters, or Acusol® 445 from Rohm and Haas. The chelant can be present in the composition in an amount of about 0.001 wt. % to about 1 wt. % by weight.

Although the compositions and hair styling products disclosed herein are directed to low- to no-VOC polymeric compositions for hair styling, there may be a need to add some solvent or co-solvent to the compositions, for example to adjust physical parameters/consumer benefits or to adapt the base formulation (polymers, water, polyol composition) to a specific product form (liquid, gel, pump, aerosol, etc.). For example, it may be necessary to add small amounts (e.g., less than 1 wt. % or so) of VOC contributing solvents to the composition to help optimize the viscosity, stability, feel, dry time, etc., to the product. In addition to adding water, the primary solvent, one can add alkanol solvents such as ethanol, propanol, isopropanol, butanol, and the like, or mono- and/or dialkylether solvents such as propyleneglycol monomethyl ether or ethylene glycol monophenyl or monobutyl ether, and the like, to these compositions. Additionally, non-VOV solvents, such as acetone, methyl acetate, d-limonene and the like may find use in the compositions. The water in the composition can be present in an amount of about 1 wt. % to about 90 wt. %, the VOC-contributing solvents at less than 2 wt. %, and the non-VOC contributing solvents or co-solvents can be present in an amount of about 1 wt. % to about 80 wt. %. Some ethanol may still be present in the final compositions due to incorporation through some of the raw materials, but it is desirable to eliminate most of the ethanol from the polymeric raw materials and not to purposely add any additional VOC contributing solvents unless the total VOC of the composition can still be kept below 2 wt. %.

The hair styling compositions and products disclosed herein comprising a combination of polymers resulting in an unexpected synergy between the polymers thereby providing an increase in hair styling properties of the hair styling products such as an increase in the hold attribute over other combinations of styling polymers. The hair styling compositions and products formed therefrom as disclosed herein can comprise a combination of three polymers including polyvinylypyrrolidone (PVP), a vinylpyrrolidone/dimethylamino propylacrylamide (DMAPA) acrylates copolymer, and/or a vinyl caprolactam/vinylpyrrolidone/dimethyaminoethyl methacrylate copolymer. The combination of these polymers can result in an unexpected synergistic effect with increased hair styling attributes, such as an increase in the hold attribute as compared to hair styling products comprising other polymers. The combination of these polymers can yield a synergistic matrix thereby forming a high hold hair styling product.

### Examples

Various hair styling compositions were formulated based upon the ingredients listed in Table 1. To prepare the Formulas A, B, C, and E, the main tank was charged with distilled water and mixing began. Before additional ingredients were added, it was ensured that all ingredients were completely dispersed. The main tank temperature was held constant at 170° F. to 180° F. throughout the following additions. Sorbitol and Natrosol were slowly added. Amaze was mixed with distilled water and heated to 160° F. to 170° F. After being completely dispersed, it was added to the main tank. To make the oil phase, the following ingredients were added being sure to mix well in between each addition: Perfecta Petrolatum, Crodafos CES, Alfol 16 Alcohol, Alfol 18 Alcohol, Brij 721, and White Beeswax. This was mixed until homogenous with the temperature being maintained at 170° F. to 180° F. throughout mixing. Once the oil phase was completely homogenous, it was slowly transferred to the main tank and mixing was continued to the emulsion is homogenous. The main tank temperature was maintained at 170° F. to 180° F. for 20 minutes and the batch was then cooled down to about 130° F. to 140° F., Luviskol K90 20%, Advantage S Solution, and Styleze CC-10 were added, mixing well between each addition. The batch was then cooled to 110° F. to 115° F., at this temperature Glydant and Fragrance (Ungerer Glacial Mint TT76-00166) were added, mixing well in between each addition.

To prepare Formula D, first Premix 1 was created by adding water to beaker 1 and heating to 160° F. to 180° F., Natrosol 250 HHR CS was then added and the two were mixed until the material was completely dissolved and there were no clumps. Premix 2 was then created by adding water to beaker 2 and heating to 160° F. to 180° F., Amaze 28-1890 was then added and the two were mixed until the material was completely dispersed and there were no visible clumps. To make Premix 3, the Oil Phase, Perfecta Petrolatum was added, mixing begun, and heated to 170° F. to 180° F. The temperature was maintained at 170° F. to 180° F. throughout the following additions: Propylparaben, Perfecta Petrolatum, Crodafos CES, Alfol 16, Alfol 18, Ganex WP-660, Brij 721, and Apifil. In the main tank, water was added to the beaker to initiate mixing and heat to 170° F. to 180° F. Sorbitol and Methylparaben were then added. The contents of Premix 1 were transferred to the main beaker and mixed well. The contents of Premix 2 were then added to the main beaker and mixed well. The contents of Premix 3 were then slowly added to the main beaker and mixing was continued until the batch was homogenous. The temperature was maintained at 170° F. to 180° F. Cooling was started by adding Luviskol VA 73W to the main tank and mixing well. Neolone PE was added and mixed well, and then fragrance was added and mixed well. The finished batch was then filled at 110° F. to 115° F.

TABLE 1

| | Formula | Formula | Formula | Formula | Formula |
|---|---|---|---|---|---|
| Ingredient | A (wt. %) | B (wt. %) | C (wt. %) | D (wt. %) | E (wt. %) |
| Water, D.I. | 22.0 | 13.3 | 14.0 | 8.8 | 20.7 |
| Sorbitol 70% HS DAB | 10.0 | 10.0 | 10.0 | 10.0 | 10.0 |
| Natrosol 250 HR | 0.25 | 0.50 | — | — | 0.1 |
| Water, D.I. | 21.85 | 25.0 | 31.0 | 10.0 | 10.0 |
| Amaze | 1.0 | 1.0 | 1.0 | 0.5 | 0.5 |
| Perfecta Petrolatum | 2.0 | 2.0 | 2.0 | 2.0 | 2.0 |
| Crodafos CES | 1.5 | 1.0 | 1.0 | 1.5 | 1.5 |
| Alfol 16NF | 0.25 | 0.25 | 1.5 | 0.75 | 0.75 |
| Alfol 18 Alcohol | 0.25 | 1.45 | 0.25 | 0.75 | 0.75 |
| Steareth-20 | 0.25 | 1.50 | 1.5 | — | |
| Beeswax | 0.50 | 0.10 | 0.1 | — | 0.3 |
| Luviskol K 9-20% | 17.5 | 25.38 | 15.0 | 20.0 | 30.0 |
| ADVANTAGE S solution | 15.0 | 9.0 | 9.0 | 12.0 | 12.0 |
| Styleze CC-10 | 7.0 | 8.87 | 13.0 | 10.0 | 10.0 |
| Parfum Glacial Mint | 0.35 | 0.35 | 0.35 | 0.35 | 0.35 |
| Glydant | 0.30 | 0.30 | 0.30 | 0.30 | 0.30 |
| Methylparaben | — | — | | 0.13 | |
| Cellosize Polymer PCG-10 | — | — | | 0.30 | |
| Propylparaben | — | — | | 0.03 | |
| Anatron WP-660 | — | — | | 1.5 | |
| Steareth-21 | — | — | | 0.75 | 0.75 |
| Apifil | — | — | | 0.3 | |
| Luviskol VA 73 W | — | — | | 10.0 | |
| Kathon CG | — | — | | 0.05 | |

Styling Products, by definition, is a product designed for the purpose of enhancing the hair's volume, hold, texture, shine, and style. Since the FDA considers styling product claims to be cosmetic, the method for measuring the efficacy of products in this category is not outlined in a monograph. The following examples used a trained hairstylist judge who provided data to determine comparative analysis of styling products. The primary objective was to evaluate the claims of the test products on male/female hair subjects at specified application intervals. Potential subjects who had completed a hair evaluation were screened by an Expert Hair Stylist for length of hair, texture of hair, chemically treated hair, and hair type. Subjects who had good quality short to long length hair joined in the study. The study followed a test designed with comparing Formulas A through E against one another in various combinations: D vs. A, D vs. B, D vs. C, B vs. C and E vs. B. Subjects were randomly assigned and half head studies were completed using one formula on one side of the head and another, different formula on the other side of the head. Applications were conducted for 1 day. One Hair Judge evaluated subjects during, after, and at the end of the day. Five subjects were assigned to test each product. Five male/female subjects ages 18-65, enrolled in the study and five completed the study. There were no adverse events reported during the course of the study.

Tables 2 through 6 illustrate the results from the various tests conducted including ease of distribution, consistency in hands, spiky, flaking, characteristics of application, all day hold, and hold. Tests were evaluated on a grade scale, system from 1-6 with 1 being bad and 6 being good.

TABLE 2
D vs. A

| Attribute | Formula D | Formula A |
|---|---|---|
| Ease of Distribution | 4 | 5 |
| Consistency in Hands | 4.5 | 6 |
| Spiky | 6 | 6 |
| Flaking | 4 | 4 |
| Characteristics | 4 | 6 |
| of Application | | |
| All Day Hold | 4 | 5 |
| Hold | 4.5 | 5 |

TABLE 3
D vs. B

| Attribute | Formula D | Formula B |
|---|---|---|
| Ease of Distribution | 4 | 6 |
| Consistency in Hands | 4.5 | 6 |
| Spiky | 6 | 6 |
| Flaking | 4.5 | 5 |
| Characteristics | 5 | 6 |
| of Application | | |
| All Day Hold | 5.5 | 5.5 |
| Hold | 6 | 6 |

TABLE 4
D vs. C

| Attribute | Formula D | Formula C |
|---|---|---|
| Ease of Distribution | 4 | 6 |
| Consistency in Hands | 4.5 | 6 |
| Spiky | 6 | 4.5 |
| Flaking | 6 | 6 |
| Characteristics | 6 | 4 |
| of Application | | |
| All Day Hold | 6 | 5 |
| Hold | 6 | 5 |

TABLE 5
B vs. C

| Attribute | Formula B | Formula C |
|---|---|---|
| Ease of Distribution | 6 | 6 |
| Consistency in Hands | 6 | 6 |
| Spiky | 6 | 4.5 |
| Flaking | 6 | 6 |
| Characteristics | 6 | 4 |
| of Application | | |
| All Day Hold | 6 | 5 |
| Hold | 6 | 4 |

TABLE 6
E vs. B

| Attribute | Formula E | Formula B |
|---|---|---|
| Ease of Distribution | 4.5 | 6 |
| Consistency in Hands | 4.5 | 6 |
| Spiky | 6 | 6 |
| Flaking | 6 | 6 |
| Characteristics | 4.5 | 6 |
| of Application | | |
| All Day Hold | 6 | 6 |
| Hold | 6 | 6 |

Results showed that Formula B had overall better performance results than A, C, D, or E. Formulas A, B and C showed that they were all easier to squeeze out of the tubes when compared to formula D and E. Formulas A, B, and C also showed that they were all easier to distribute throughout the hair when compared to Formula D. All formulas A, B, and C were creamier in consistency. Formula B was creamy, but showed that it was a little thicker than Formulas A and C. Formula B also showed that it had a little more tack and hold in the hands comparable to Formulas D and E.

In an embodiment, a hair styling composition comprises: 5 wt. % to 50 wt. % of a first nonionic polymer; 2 wt. % to 20 wt. % of a second nonionic polymer; and 2 wt. % to 15 wt. % of a cationic polymer.

In an embodiment, a method of making a hair styling composition comprises: combining 5 wt. % to 50 wt. % of a first nonionic polymer, 2 wt. % to 20 wt. % of a second nonionic polymer, and 2 wt. % to 15 wt. % of a cationic polymer to form a hair styling composition.

In the various embodiments, (i) the first and/or the second nonionic polymer is selected from the group consisting of polyvinylpyrrolidone, copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, copolymers of vinylpyrrolidone and dimethylamino propylacrylamide acrylate, polysiloxanes, and combinations comprising at least one of the foregoing; and/or (ii) the first nonionic polymer is polyvinylpyrrolidone; and/or (iii) the second nonionic polymer is vinylpyrrolidone dimethlyamino propylacrylamide acrylate copolymer; and/or (iv) the cationic polymer is selected from the group consisting of vinylpyrrolidone/vinylimidazolium methochloride copolymers, quaternized vinylpyrrolidone/diakylaminoalkyl methacrylate copolymers, cationic cellulose derivatives, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers, and combinations comprising at least one of the foregoing; and/or (v) the cationic polymer is a terpolymer of vinylcaprolactam/vinylpyrrolidone with dimethylaminoethyl methacrylate; and/or (vi) the first nonionic polymer is present in an amount of 15 wt. % to 30 wt. %; and/or (vii) the second anionic polymer is present in an amount of 8 wt. % to 13 wt. %; and/or (viii) the cationic polymer is present in an amount of 9 wt. % to 12 wt. %; and/or (ix) the composition has a higher hold attribute compared to a hair styling composition comprising a greater number of polymers; and/or (x) an article comprises the hair styling composition; and/or (xi) the article is a hair styling product.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1. A hair styling composition, comprising:
5 wt. % to 50 wt. % of a first nonionic polymer;
2 wt. % to 20 wt. % of a vinylpyrrolidone dimethylamino propylacrylamide acrylate copolymer; and
2 wt. % to 15 wt. % of a cationic polymer.

2. The hair styling composition of claim 1, wherein the first nonionic polymer is selected from the group consisting of polyvinylpyrrolidone, copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, polysiloxanes, and combinations comprising at least one of the foregoing.

3. The hair styling composition of claim 2, wherein the first nonionic polymer is polyvinylpyrrolidone.

4. (canceled)

5. The hair styling composition of claim 1, wherein the cationic polymer is selected from the group consisting of vinylpyrrolidone/vinylimidazolium methochloride copolymers, quaternized vinylpyrrolidone/diakylaminoalkyl methacrylate copolymers, cationic cellulose derivatives, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers, and combinations comprising at least one of the foregoing.

6. The hair styling composition of claim 5, wherein the cationic polymer is a terpolymer of vinylcaprolactam/vinylpyrrolidone with dimethylaminoethyl methacrylate.

7. The hair styling composition of claim 1, wherein the first nonionic polymer is present in an amount of 15 wt. % to 30 wt. %.

8. The hair styling composition of claim 1, wherein the second anionic polymer is present in an amount of 8 wt. % to 13 wt. %.

9. The hair styling composition of claim 1, wherein the cationic polymer is present in an amount of 9 wt. % to 12 wt. %.

10. The hair styling composition of claim 1, wherein the composition has a higher hold attribute compared to a hair styling composition comprising a greater number of polymers.

11. An article comprising the hair styling composition of claim 1.

12. The article of claim 11, wherein the article is a hair styling product.

13. A method of making a hair styling composition, comprising:
combining 5 wt. % to 50 wt. % of a first nonionic polymer, 2 wt. % to 20 wt. % of a second nonionic polymer, and 2 wt. % to 15 wt. % of a cationic polymer to form a hair styling composition.

14. The hair styling composition of claim 13, wherein the first and/or the second nonionic polymer is selected from the group consisting of polyvinylpyrrolidone, copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, copolymers of vinylpyrrolidone and dimethylamino propylacrylamide acrylate, polysiloxanes, and combinations comprising at least one of the foregoing.

15. The hair styling composition of claim 14, wherein the first nonionic polymer is polyvinylpyrrolidone and wherein the second nonionic polymer is vinylpyrrolidone dimethlyamino propylacrylamide acrylate copolymer.

16. The hair styling composition of claim 13, wherein the cationic polymer is selected from the group consisting of vinylpyrrolidone/vinylimidazolium methochloride copolymers, quaternized vinylpyrrolidone/diakylaminoalkyl methacrylate copolymers, cationic cellulose derivatives, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers, and combinations comprising at least one of the foregoing.

17. The hair styling composition of claim 16, wherein the cationic polymer is a terpolymer of vinylcaprolactam/vinylpyrrolidone with dimethylaminoethyl methacrylate.

18. The hair styling composition of claim 13, wherein the first nonionic polymer is present in an amount of 15 wt. % to 30 wt. %.

19. The hair styling composition of claim 13, wherein the second anionic polymer is present in an amount of 8 wt. % to 13 wt. %.

20. The hair styling composition of claim 13, wherein the cationic polymer is present in an amount of 9 wt. % to 12 wt. %.
